(19) United States
(12) Patent Application Publication
(10) Pub. No.: US 2006/0009845 A1
(43) Pub. Date: Jan. 12, 2006

(54) Method and device for kinematic retaining cervical plating

(76) Inventor: Kingsley R. Chin, Philadelphia, PA (US)

Correspondence Address: AKC PATENTS, 215 GROVE ST., NEWTON, MA, 02466, US

(21) Appl. No.: 11/174,712

(22) Filed: Jul. 5, 2005

Related U.S. Application Data
(60) Provisional application No. 60/586,761, filed on Jul. 8, 2004.

(51) Int. Cl. A61F 2/44; A61B 17/70

(52) U.S. Cl. 623/17.11; 623/17.15; 606/71

(57) ABSTRACT

A spinal implant assembly for replacing intervertebral elements between a first spinal vertebra and an adjacent second spinal vertebra includes an intervertebral implant for inserting between the first and second spinal vertebrae and a first kinematic retaining plate. The intervertebral implant comprises a body having a top surface, a bottom surface, and a first appendage extending from the top surface of the intervertebral implant. The first appendage is adapted to fit within and form a tongue and groove attachment with a first opening formed in the first spinal vertebra. The first kinematic retaining plate is attached to the first spinal vertebra so that it secures the first appendage in the first opening. The intervertebral implant further comprises a second appendage extending from the bottom surface and the second appendage is adapted to fit within and form a tongue and groove attachment with a second opening formed in the second spinal vertebra. A second kinematic retaining plate is attached to the second spinal vertebra so that it secures the second appendage in the second opening.

### CROSS REFERENCE TO RELATED CO-PENDING APPLICATIONS

This application claims the benefit of U.S. provisional application Ser. No. 60/586,761 filed on Jul. 8, 2004 and entitled METHODS AND DEVICES FOR KINEMATIC RETAINING CERVICAL (KRC) PLATING which is commonly assigned and the contents of which are expressly incorporated herein by reference.

### FIELD OF THE INVENTION

The present invention relates to an apparatus and a method for connecting and stabilizing spinal vertebrae, and more particularly to an apparatus and a method that connects spinal vertebrae while preserving spinal stability and mobility.

### BACKGROUND OF THE INVENTION

The human spine 29 comprises individual vertebras 30 (segments) that are connected to each other to form a spinal column, shown in FIG. 1A. The vertebras 30 are separated and cushioned by thin pads of tough, resilient fiber known as inter-vertebral discs 40, shown in FIG. 1B. Inter-vertebral discs 40 provide flexibility to the spine 29 and act as shock absorbers during activity. The function of the spine 29 is to protect the neural structures 44 and to allow us to stand erect, bear axial loads, and be flexible for bending and rotation. Disorders of the spine occur when one or more of the individual vertebras 30 and/or the inter-vertebral discs 40 are abnormal. In these pathologic circumstances, surgery may be tried to restore the function of the spine to normal, achieve stability, protect the neural structures 44, or to relief the patient of discomfort. The goal of spine surgery for a multitude of spinal disorders especially those causing compression of the neural structures 44 is often decompression of the neural elements and/or fusion of adjacent vertebral segments. Fusion works well because it stops pain due to movement at the facet joints or intervertebral discs, holds the spine in place after correcting deformity, and prevents instability and or deformity of the spine after spine procedures such as laminectomies or corpectomies. Laminectomy involves the removal of part of the lamina 47, i.e., the bony roof of the spinal canal, shown in FIG. 1C. Corpectomy involves removal of the vertebral body 32 as well as the adjacent disc spaces 40. Laminectomy is often used to directly decompress the posterior neural elements 42 and to relieve pain or neurologic compromise caused by posterior compressive structures. In some cases laminectomy may also achieve indirect decompression of anterior compressive structures.

In contrast, anterior decompression directly removes anterior compressive structures and is known to have improved results in these cases over indirect decompression afforded by laminectomies. Anterior discectomy, i.e., removal of the inter-vertebral discs 40, and fusion or anterior corpectomy and fusion are most commonly performed in the cervical spine but there is increasing application in the thoracic and lumbar spine.

In recent years, there is an increase in the use of plate fixation 27 to stabilize the cervical spine 28 after anterior decompression and fusion, shown in FIG. 1D. Plate fixation 27 provides increased stability and may allow for less reliance on rigid external orthosis such as hard cervical collars and halos for stability. Plates 27 may also increase the rate of fusion and may decrease the incidence of graft complications such as graft extrusions and subsidence. Although, current plating systems offer these advantages, there is a growing body of data that document significant failure rates for reconstruction with plates after multilevel anterior corpectomy and fusion. It is believed that the long lever arm of the plate especially across two or more vertebral corpectomies leads to pullout of the screws 25 and dislodgement of the plate 27 which can result in esophageal erosion and death. Furthermore, current anterior cervical plates 27 do not provide graft subsidence and continuous graft loading which is believed to be advantageous for fusion. It is also technically challenging to place a plate 27 across two or more disc spaces while maintaining the correct length and avoiding placing the screws 25 in the graft or the adjacent disc space. It is also difficult to place the plate 27 in a straight line longitudinally between adjacent vertebras. These technical difficulties often lead to a higher rate of complications including plate failures.

A modification of the standard cervical plate has been tried to function as a buttress plate. In the buttress plate design, the plate is attached to one vertebral body and extends across the endplate and partially over the graft to act solely as a block to graft dislodgement. However, this design has been abandoned since it was demonstrated that these buttress plates would dislodge when the graft shifted anteriorly against the plate and would themselves cause catastrophic problems such as esophageal erosions. Part of the reason for this failure is the fact that the plates were designed to overhang the disc space, which created a lever arm that made it easier to dislodge the anchor screws 25 in the vertebral body.

More recently, plates have been designed to allow motion between the fused segments either at the fixation points between the plate 27 and the screws 25 or as a sliding mechanism within the plate with the ends of the plate fixed to screws in the vertebral body. Examples of these “dynamic” plating systems include the Ant-Cer system offered by Spinal Concepts of Texas, and the ABC system offered by Aesculap, of Germany. These new “dynamic” plating systems are believed to offer superior fusion rates since they allow continuous graft loading and natural graft subsidence while acting as a block to anterior graft displacement. However, these new “dynamic” plating systems still do not remove the technical difficulties in placing the plate across the entire length of the fused segments.

Accordingly, there is a need for a plating system that removes the difficulties in placing the plate across the entire length of the fused segments, while providing stability and allowing motion between the fused segments.

### SUMMARY OF THE INVENTION

In general, in one aspect, the invention features a spinal implant assembly for replacing intervertebral elements between a first spinal vertebra and an adjacent second spinal vertebra. The spinal implant assembly includes an intervertebral implant for inserting between the first and second spinal vertebrae and a first kinematic retaining plate. The intervertebral implant comprises a body having a top surface, a bottom surface, and a first appendage extending from the top surface of the intervertebral implant. The first appendage is adapted to fit within and form a tongue and groove attachment with a first opening formed in the first spinal vertebra. The first kinematic retaining plate is attached to the first spinal vertebra so that it secures the first appendage in the first opening. The intervertebral implant further comprises a second appendage extending from the bottom surface and the second appendage is adapted to fit within and form a tongue and groove attachment with a second opening formed in the second spinal vertebra. A second kinematic retaining plate is attached to the second spinal vertebra so that it secures the second appendage in the second opening.

Implementations of this aspect of the invention may include one or more of the following features. The first kinematic plate has on or more holes and is attached to the first spinal vertebra via one or more screws going through the one or more holes, respectively. The first appendage comprises side surfaces that are straight, curved, serrated, spiked, or angled relative to the top surface of the intervertebral implant, and the first opening comprises corresponding side surfaces that are straight, curved, serrated, spiked or angled relative to the top surface of the intervertebral implant, respectively. The vertebrae are cervical vertebrae, thoracic vertebra, or lumbar vertebrae. The intervertebral implant is made of bone, polyetheretherketone (PEEK), Nitinol, metals, titanium, steel, metal composites, biodegradable materials, collagen matrices, synthetic polymers, polysaccharides, calcium minerals, calcium salts, or composites containing calcium or phosphorous naturally or man made. The kinematic retaining plate is made of bone, polyetheretherketone (PEEK), Nitinol, metals, titanium, steel, metal composites, biodegradable materials, or composites containing calcium or phosphorous naturally or man made. The intervertebral implant comprises more than one appendages extending from the top surface, and the more than one appendages are adapted to fit within and form tongue and groove attachments with more than one openings formed in the first spinal vertebra. The intervertebral implant further comprises one or more cavities or one or more fenestrations. The intervertebral implant comprises an elastic structure. The intervertebral implant is inserted between the first and second spinal vertebrae for providing either anterior spinal fusion or posterior spinal fusion.

In general, in another aspect, the invention features a spinal implant assembly for replacing intervertebral elements between a first spinal vertebra and an adjacent second spinal vertebra. The spinal implant assembly comprises an intervertebral implant for inserting between the first and second spinal vertebrae, the intervertebral implant comprising a body having a top surface, a bottom surface, and first and second appendages extending from the top surface and the bottom surface, respectively. The first and the second appendages are adapted to fit within and form a tongue and groove attachment with first and second openings formed in the first and second spinal vertebrae, respectively. The first and the second appendages comprise first and second holes, respectively, and are attached to the first and second spinal vertebrae via first and second screws going through the first and second holes, respectively.

In general, in another aspect, the invention features a spinal implant assembly for replacing intervertebral elements between a first spinal vertebra and an adjacent second spinal vertebra. The spinal implant assembly comprises first and second intervertebral implants for inserting between the first and second spinal vertebrae. The first intervertebral implant comprises a body having a top surface, a bottom surface, and a first appendage extending from the top surface. The first appendage is adapted to fit within and form a tongue and groove attachment with a first opening formed in the first spinal vertebra. The second intervertebral implant comprises a body having a top surface, a bottom surface, and a second appendage extending from the bottom surface. The second appendage is adapted to fit within and form a tongue and groove attachment with a second opening formed in the second spinal vertebra. The first and the second appendages comprise first and second holes, respectively, and are further attached to the first and second spinal vertebra via first and second screws going through the first and second holes, respectively.

Implementations of this aspect of the invention may include one or more of the following features. The bottom surface of the first intervertebral implant comprises a first articulating structure and the top surface of the second intervertebral implant comprises a second articulating structure configured to articulate with the first articulating structure. The first intervertebral implant is articulately connected to the second intervertebral implant by articulating the first and the second articulating structures. The bottom surface of the first intervertebral implant and the top surface of the second intervertebral implant comprise coatings made of titanium, tantalum, stainless steel, polyethylene, diamond, chrome, cobalt, biodegradable materials, metal alloys, ceramic, or composites.

In general, in another aspect, the invention features method of replacing intervertebral elements between a first spinal vertebra and an adjacent second spinal vertebra. The method includes inserting an intervertebral implant between the first and second spinal vertebrae. The intervertebral implant comprises a body having a top surface, a bottom surface, and a first appendage extending from the top surface of the intervertebral implant. The first appendage is adapted to fit within and form a tongue and groove attachment with a first opening formed in the first spinal vertebra. The method also includes attaching a first kinematic retaining plate to the first spinal vertebra so that it secures the first appendage in the first opening.

Among the advantages of this invention may be one or more of the following. The implantable graft and kinematic retaining plates stabilize the spine, while allowing the patient to retain spinal flexibility by preserving motion between adjacent vertebras. The design of the plates allows for easy placement of the plates and screws because the plates are attached to only one vertebral body. The tongue and groove attachment configuration between the graft and the vertebral bodies provides more surfaces for better fusion between the graft and the endplates of the vertebras and greater stability for rotation. Furthermore, because the plates are confined to the vertebral bodies, this design allows for stability of the ends of the graft while allowing for natural graft subsidence and dynamic graft loading of the remainder of he graft and while preventing graft dislodgement.

The details of one or more embodiments of the invention are set forth in the accompanying drawings and description below. Other features, objects and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1A is a side view of the human spinal column;

FIG. 1B is an enlarged view of area A of FIG. 1A;

FIG. 1C is an axial cross-sectional view of a vertebra;

FIG. 1D is a radiographic side view of a cervical plating system;

FIG. 2 is a schematic view of the process of removing an intervertebral disc and inserting a graft between two vertebras;

FIG. 3A is a schematic view of the process of securing the graft of FIG. 2 by attaching two kinematic retaining plates;

FIG. 3B is a side cross-sectional view (along axis AA′) of the spinal implant assembly of FIG. 3A;

FIG. 4 is a perspective schematic view of a vertebra with resected vertebral body;

FIG. 5 depicts schematic diagrams of various graft shapes;

FIG. 6A depicts another embodiment of the spinal implant assembly;

FIG. 6B is a side cross-sectional view (along axis AA′) of the embodiment of FIG. 6A;

FIG. 7A depicts another embodiment of the spinal implant assembly;

FIG. 7B is a side cross-sectional view (along axis AA′) of the embodiment of FIG. 7A;

FIG. 8A depicts another embodiment of the spinal implant assembly; and

FIG. 8B is a side cross-sectional view (along axis AA′) of the embodiment of FIG. 8A.

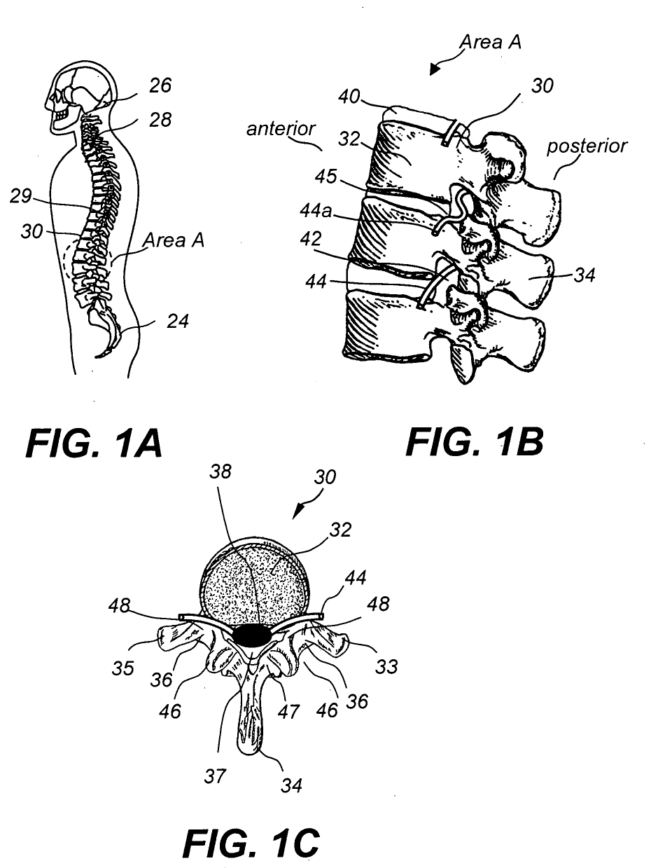

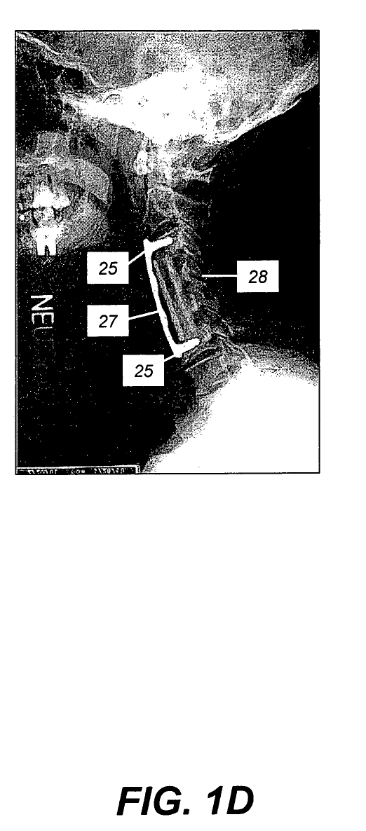

### DETAILED DESCRIPTION OF THE INVENTION

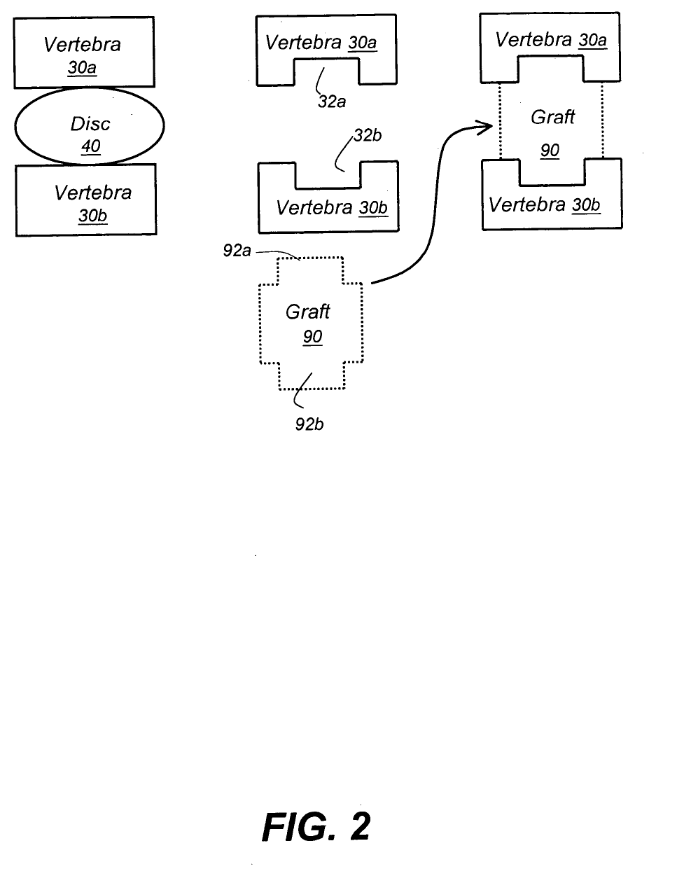

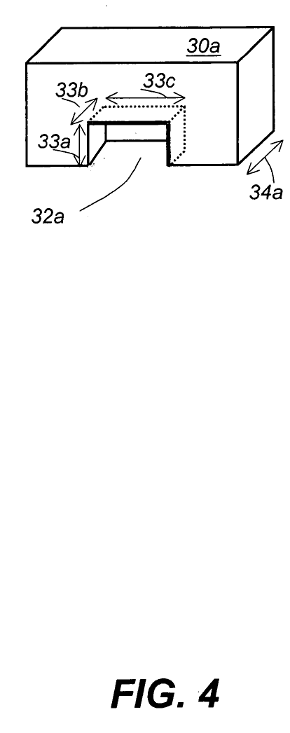

Referring to FIG. 2, a new grafting technique for replacing an intervertebral disc 40 includes first removing the intervertebral disc 40 form the space between two adjacent vertebras 30a, 30b, then forming grooves 32a, 32b in vertebras 30a, 30b, respectively, then preparing a graft 90 and inserting the graft in the space between the vertebras 30a, 30b. The graft 90 is either an autograft or an allograft and includes tongue extensions 92a, 92b extending from the top 91a and bottom 91b of the graft 90, respectively. The tongue extensions 92a, 92b are designed to fit closely in grooves 32a, 32b, respectively, in a tongue and groove or “dovetail” attachment configuration. The tongue and groove attachment configuration provides multidirectional stability and allows immediate range of motion of the spine without the need for external bracing. In one example, shown in FIG. 4, the groove 32a, has dimensions 33a, 33b, 33c of 3 mm, 10 mm, 5 mm, respectively. The dimension 33b is usually less than the dimension 34a of the vertebra 30a. The tongue extension 92a has a similar three dimensional configuration as the groove 32a and is dimensioned to fit closely within the groove 32a. Grooves 32a, 32b are formed within the vertebras 30a, 30b, respectively, with a special instrument. In one example, this special instrument is a burr with a stop that allows the formation of a groove with a predetermined depth. In another example, this special instrument is a cutting device with a stop that allows the formation of a groove with a predetermined depth and shape.

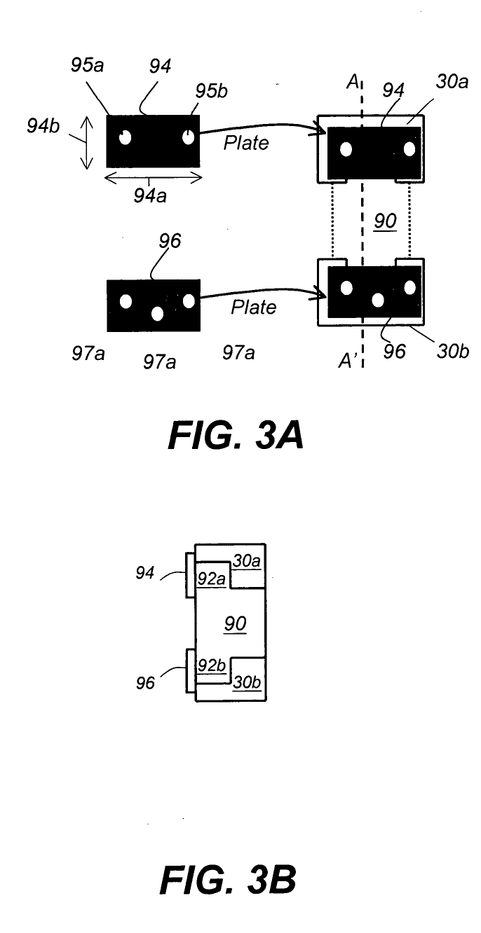

Referring to FIGS. 3A and 3B, kinematic retaining plates 94 and 96 are placed over and attached to the vertebras 30a, 30b, respectively. Plates 94, 96 prevent the dislodgment of the graft 90 while allowing dynamization of the graft, since they do not restrict vertical motion. In one example, plates 94, 96 have rectangular shape and have dimensions 94a of 14 mm and 94b of 5 mm. Plate 94 includes two screw holes 95a, 95b, and plate 96 includes three screw holes 97a, 97b, 97c. Holes 95a, 95b and 97a, 97b, 97c allow fixed or variable angled screws to be inserted into the vertebral bodies of vertebras 30a, 30b, respectively for attaching the plates to the vertebras.

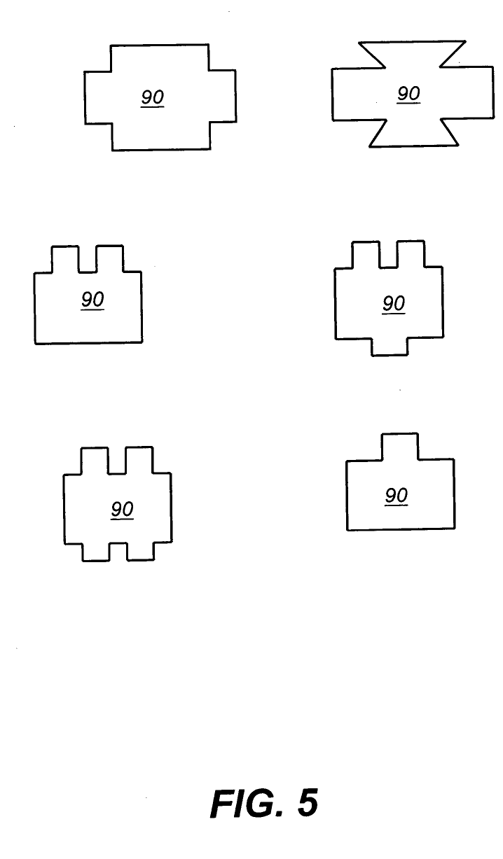

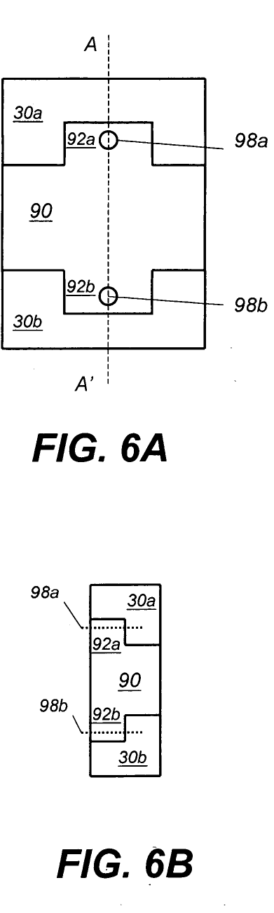

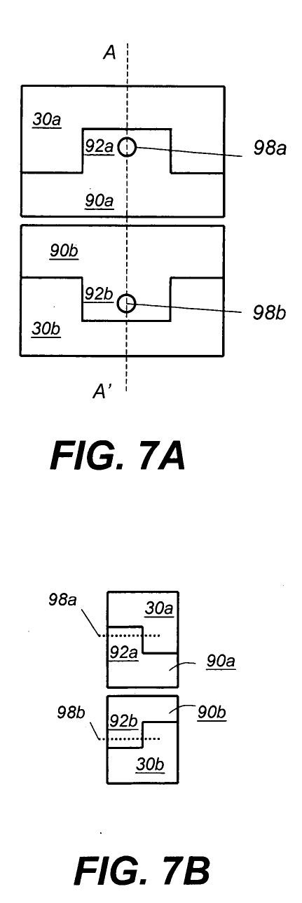

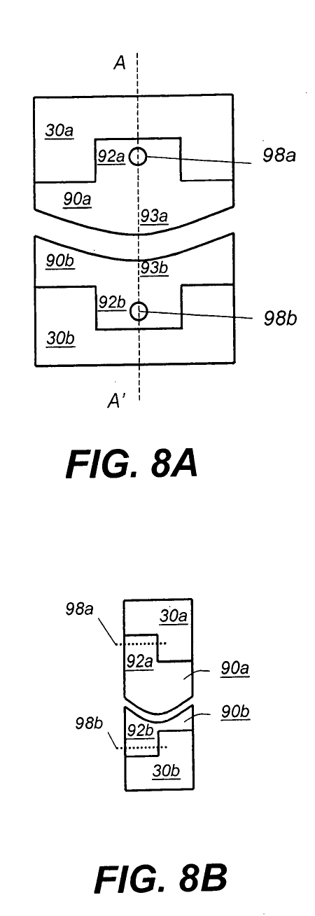

Other embodiments are within the scope of the following claims. Retaining plates 94, 96 may be circular, oblong, have rounded edges, or have multiple screw holes. One or more screws may go through the plate and any part of the graft in order to attach the graft to the plate. The graft and plate may be one-piece such that the plate acts as a stop against the vertebral body. Referring to FIG. 6A and FIG. 6B, the tongue extensions 92a, 92b, may include holes 98a, 98b respectively, that receive screws for attaching the graft directly to the vertebras 30a, 30b. In this embodiment there is no need for a plate to further secure the graft to the vertebral bodies. This configuration allows even more stability to the construct. In another embodiment, the graft tongue extensions 92a, 92b, may have front surfaces (not shown), that overhang and extend to cover the front of the vertebral openings 32a, 32b. Also, by graft we mean any one-piece interbody structure that has a design that interdigitates with the vertebras in a tongue and groove attachment form, as described above. The graft may be made of bone, polyetheretherketone (PEEK), Nitinol, metal such as titanium, steel, or metal composites, biodegradable material, composites containing calcium or phosphorous naturally or man made. The graft may be solid or have one or more cavities that are enclosed or open or one or more fenestrations. Referring, to FIG. 5, there may be one or more tongue extensions extending from the top or bottom surfaces of the graft to interdigitate with the vertebral endplates either straight or angled from 0 to 90 degrees with the surface of the vertebral endplates. The surface of the tongue may comprise of straight sides with or without serrations or “spikes”. The shape of the tongue extensions may also vary to have angled or curved surfaces. The graft may be expandable or compressible either through the material properties such as Nitinol or mechanically. The tongue and groove relationship between the graft and the vertebral endplate may be with one or both vertebral endplates. The graft can be one piece connecting between the two adjacent vertebral endplates or two separate pieces 90a, 90b with a space between the ends opposite to the ends connected to the vertebral endplates, as shown in FIG. 7A, and FIG. 7B. The space between the grafts 90a, 90b allows for multidirectional motion. The ends of the graft may be covered with materials of varying properties and durability that include but not limited to titanium, stainless steel, polyethylene, diamond, chrome, cobalt, biodegradable materials, metal alloys. These surface coverings may be capped or coated on the ends of the graft. The adjacent ends 93a, 93b of the two separate intervertebral pieces 90a, 90b, respectively, may include articulating structures, as shown in FIG. 9A and FIG. 9B. The articulating structures may have varying configuration from a flat on flat design to a ball and socket design, as shown in FIG. 9A, and FIG. 9B. This design is the first to combine a graft material that may fuse to the endplate and that is contained within the endplate by a plate as described in this application and also having a different -material covering the opposite end that allows for articulation between vertebral endplates secondarily to articulation between the ends of the graft. Other motion preserving designs such as disc replacements have a modular polyethylene core between two connecting end pieces or have two articulating pieces that are also connected to the endplates as a single piece.

Several embodiments of the present invention have been described. Nevertheless, it will be understood that various modifications may be made without departing from the spirit and scope of the invention. Accordingly, other embodiments are within the scope of the following claims.

What is claimed is:

1. A spinal implant assembly for replacing intervertebral elements between a first spinal vertebra and an adjacent second spinal vertebra comprising:
an intervertebral implant for inserting between said first and second spinal vertebrae, said intervertebral implant comprising a body having a top surface, a bottom surface, and a first appendage extending from said top surface of said intervertebral implant, and wherein said first appendage is adapted to fit within and form a tongue and groove attachment with a first opening formed in said first spinal vertebra; and
a first kinematic retaining plate adapted to be attached to said first spinal vertebra so that it secures said first appendage in said first opening.

2. The spinal implant assembly of claim 1 wherein said intervertebral implant further comprises a second appendage extending from said bottom surface and said second appendage is adapted to fit within and form a tongue and groove attachment with a second opening formed in said second spinal vertebra.

3. The spinal implant assembly of claim 2 further comprising a second kinematic retaining plate adapted to be attached to said second spinal vertebra so that it secures said second appendage in said second opening.

4. The spinal implant assembly of claim 1 wherein said first kinematic plate comprises on or more holes and is attached to said first spinal vertebra via one or more screws going through said one or more holes, respectively.

5. The spinal implant assembly of claim 1 wherein said first appendage comprises side surfaces selected from a group consisting of straight, curved, serrated, spiked, and angled relative to said top surface of said intervertebral implant, and wherein said first opening comprises corresponding side surfaces selected from a group consisting of straight, curved, serrated, spiked and angled relative to said top surface of said intervertebral implant, respectively.

6. The spinal implant assembly of claim 1 wherein said vertebrae are selected from a group consisting of cervical vertebrae, thoracic vertebra, and lumbar vertebrae.

7. The spinal implant assembly of claim 1 wherein said intervertebral implant is made of a material selected from a group consisting of bone, polyetheretherketone (PEEK), Nitinol, metals, titanium, steel, metal composites, biodegradable materials, collagen matrices, synthetic polymers, polysaccharides, calcium minerals, calcium salts, and composites containing calcium or phosphorous naturally or man made.

8. The spinal implant assembly of claim 1 wherein said kinematic retaining plate is made of a material selected from a group consisting on bone, polyetheretherketone (PEEK), Nitinol, metals, titanium, steel, metal composites, biodegradable materials, and composites containing calcium or phosphorous naturally or man made.

9. The spinal implant assembly of claim 1 wherein said intervertebral implant comprises more than one appendages extending from said top surface, and wherein said more than one appendages are adapted to fit within and form tongue and groove attachments with more than one openings formed in said first spinal vertebra.

10. The spinal implant assembly of claim 1 wherein said intervertebral implant further comprises one or more cavities.

11. The spinal implant assembly of claim 1 wherein said intervertebral implant further comprises one or more fenestrations.

12. The spinal implant assembly of claim 1 wherein said intervertebral implant comprises an elastic structure.

13. The spinal implant assembly of claim 1 wherein said intervertebral implant is inserted between said first and second spinal vertebrae for providing anterior spinal fusion.

14. The spinal implant assembly of claim 1 wherein said intervertebral implant is inserted between said first and second spinal vertebrae for providing posterior spinal fusion.

15. A spinal implant assembly for replacing intervertebral elements between a first spinal vertebra and an adjacent second spinal vertebra comprising:
an intervertebral implant for inserting between said first and second spinal vertebrae, said intervertebral implant comprising a body having a top surface, a bottom surface, and first and second appendages extending from said top surface and said bottom surface, respectively, and wherein said first and said second appendages are adapted to fit within and form a tongue and groove attachment with first and second openings formed in said first and second spinal vertebrae, respectively ; and
wherein said first and said second appendages comprise first and second holes, respectively, and are further attached to said first and second spinal vertebra via first and second screws going through said first and second holes, respectively.

16. A spinal implant assembly for replacing intervertebral elements between a first spinal vertebra and an adjacent second spinal vertebra comprising:
a first intervertebral implant for inserting between said first and second spinal vertebrae, said first intervertebral implant comprising a body having a top surface, a bottom surface, and a first appendage extending from said top surface and wherein said first appendage is adapted to fit within and form a tongue and groove attachment with a first opening formed in said first spinal vertebra;
a second intervertebral implant for inserting between said first and second spinal vertebrae, said second intervertebral implant comprising a body having a top surface, a bottom surface, and a second appendage extending from said bottom surface and wherein said second appendage is adapted to fit within and form a tongue and groove attachment with a second opening formed in said second spinal vertebra; and
wherein said first and said second appendages comprise first and second holes, respectively, and are further attached to said first and second spinal vertebra via first and second screws going through said first and second holes, respectively.

17. The spinal implant assembly claim 16 wherein said bottom surface of said first intervertebral implant comprises a first articulating structure and said top surface of said second intervertebral implant comprises a second articulating structure configured to articulate with said first articulating structure, and wherein said first intervertebral implant is articulately connected to said second intervertebral implant by articulating said first and said second articulating structures.

18. The spinal assembly of claim 16, wherein said bottom surface of said first intervertebral implant and said top surface of said second intervertebral implant comprise materials selected from a group consisting of titanium, tantalum, stainless steel, polyethylene, diamond, chrome, cobalt, biodegradable materials, metal alloys, ceramic, and composites.

19. A method of replacing intervertebral elements between a first spinal vertebra and an adjacent second spinal vertebra comprising:
inserting an intervertebral implant between said first and second spinal vertebrae, wherein said intervertebral implant comprises a body having a top surface, a bottom surface, and a first appendage extending from said top surface of said intervertebral implant, and wherein said first appendage is adapted to fit within and form a tongue and groove attachment with a first opening formed in said first spinal vertebra; and
attaching a first kinematic retaining plate to said first spinal vertebra so that it secures said first appendage in said first opening.

20. The method of claim 19 further comprising removing said intervertebral elements before inserting said intervertebral implant.

21. The method of claim 19 wherein said intervertebral implant further comprises a second appendage extending from said bottom surface and said second appendage is adapted to fit within and form a tongue and groove attachment with a second opening formed in said second spinal vertebra.

22. The method of claim 21 further comprising attaching a second kinematic retaining plate to said second spinal vertebra so that it secures said second appendage in said second opening.

23. The method of claim 19 wherein said first kinematic plate comprises on or more holes and is attached to said first spinal vertebra via one or more screws going through said one or more holes, respectively.

24. The method of claim 19 wherein said first appendage comprises side surfaces selected from a group consisting of straight, curved, serrated, spiked, and angled relative to said top surface of said intervertebral implant, and wherein said first opening comprises corresponding side surfaces selected from a group consisting of straight, curved, serrated, spiked and angled relative to said top surface of said intervertebral implant, respectively.

25. The method of claim 19 wherein said vertebrae are selected from a group consisting of cervical vertebrae, thoracic vertebra, and lumbar vertebrae.

26. The method of claim 19 wherein said intervertebral implant is made of a material selected from a group consisting of bone, polyetheretherketone (PEEK), Nitinol, metals, titanium, steel, metal composites, biodegradable materials, collagen matrices, synthetic polymers, polysaccharides, calcium minerals, calcium salts, and composites containing calcium or phosphorous naturally or man made.

27. The method of claim 19 wherein said kinematic retaining plate is made of a material selected from a group consisting on bone, polyetheretherketone (PEEK), Nitinol, metals, titanium, steel, metal composites, biodegradable materials, and composites containing calcium or phosphorous naturally or man made.

28. The method of claim 19 wherein said intervertebral implant comprises more than one appendages extending from said top surface, and wherein said more than one appendages are adapted to fit within and form tongue and groove attachments with more than one openings formed in said first spinal vertebra.

29. The method of claim 19 wherein said intervertebral implant further comprises one or more cavities.

30. The method of claim 19 wherein said intervertebral implant further comprises one or more fenestrations.

31. The method of claim 19 wherein said intervertebral implant comprises elastic properties.

32. The method of claim 19 wherein said intervertebral implant is inserted between said first and second spinal vertebrae for providing anterior spinal fusion.

33. The method of claim 19 wherein said intervertebral implant is inserted between said first and second spinal vertebrae for providing posterior spinal fusion.

34. A method for replacing intervertebral elements between a first spinal vertebra and an adjacent second spinal vertebra comprising:
inserting a first intervertebral implant between said first and second spinal vertebrae, said first intervertebral implant comprising a body having a top surface, a bottom surface, and a first appendage extending from said top surface and wherein said first appendage is adapted to fit within and form a tongue and groove attachment with a first opening formed in said first spinal vertebra;
inserting a second intervertebral implant between said first and second spinal vertebrae, said second intervertebral implant comprising a body having a top surface, a bottom surface, and a second appendage extending from said bottom surface and wherein said second appendage is adapted to fit within and form a tongue and groove attachment with a second opening formed in said second spinal vertebra; and
attaching said first and said second appendages to said first and said second spinal vertebra via first and second screws going through first and second holes formed in said first and second appendages, respectively.

35. The method of claim 34 wherein said bottom surface of said first intervertebral implant comprises a first articulating structure and said top surface of said second intervertebral implant comprises a second articulating structure configured to articulate with said first articulating structure, and said method further comprising connecting said first intervertebral implant to said second intervertebral implant by articulating said first and said second articulating structures.

36. The method of claim 34, wherein said bottom surface of said first intervertebral implant and said top surface of said second intervertebral implant comprise materials selected from a group consisting of titanium, tantalum, stainless steel, polyethylene, diamond, chrome, cobalt, biodegradable materials, metal alloys, ceramic, and composites.
